(19) United States
(12) Patent Application Publication
(10) Pub. No.: US 2025/0092410 A1
(43) Pub. Date: Mar. 20, 2025

(54) NF-YA5/miR169a MODULE CONTROLLING NITROGEN UTILIZATION EFFICIENCY OF PLANT AND USES THEREOF

(71) Applicant: Lasemilla Co., Ltd., Gangwon-do (KR)

(72) Inventors: Ju-Kon KIM, Gangwon-do (KR); Jun Sung SEO, Gangwon-do (KR); Se-Jun OH, Seoul (KR)

(21) Appl. No.: 18/368,607

(22) Filed: Sep. 15, 2023

(51) Int. Cl. C12N 15/82

(52) U.S. Cl. CPC C12N 15/8243; C12N 15/8218; Y02A 40/132; Y02A 40/146

(57) ABSTRACT

A NF-YA5/miR169a module controls nitrogen utilization efficiency of plant and uses thereof. Since the NF-YA5/miR169a module enables the enhancement of the nitrogen utilization efficiency in plants, it can be advantageously used for the development of environmentally friendly and cost-effective plants that can reduce the consumption of nitrogen fertilizers while maintaining crop yields.

### BACKGROUND

### 1. Technical Field

The present invention relates to a NF-YA5/miR169a module controlling nitrogen utilization efficiency of plant and uses thereof.

### 2. Background Art

Nitrogen is one of the essential macronutrients for plants growth and plant mostly utilize nitrate (NO₃⁻), ammonium (NH₄⁺) and amino acid as nitrogen source. Nitrate is taken up by the nitrate transporter (NRT) in roots, uptake of ammonium is mediated by own transporter, ammonium transporter (AMT) and both trans-located to the shoot in the form of amino acid after assimilation process. Not only being a nutrient, nitrogen regulates nitrogen responsive gene expression, root morphological traits, floral induction, leaf senescence, and agronomic traits in rice. Consistently, leaf nitrogen content is positively correlated with chlorophyll contents and total grain yield, which are more reliable at the panicle formation stage rather than at the booting stage. Thus, application of chemical nitrogen fertilizers was widely used to support the increasing demand in foods due to the rapid growth of world population. However, plants do not take up nitrogen in the soil completely, more than 50% of nitrogen leached resulting in the eutrophication and pollution of the environment. Therefore, it is a challenge to reduce fertilizer usage while maintaining productivity and yield by improving nitrogen use efficiency (NUE). Several studies have been reported to improve the NUE of crops, focused on the genetic manipulation for nitrogen uptake, nitrogen metabolism as well as nitrogen regulation.

MicroRNAs (miRNAs) are small non-coding ribonucleic acids that regulate gene expression by cleavage mRNA target, translational repression, mRNA deadenylation via base pairing to the target mRNAs. OsmiR169 is a big microRNA family containing 17 known members representing nine different mature isoforms in rice. OsmiR169 is up-regulated in high-nitrate treatments and play a role in long-distance signaling reporting N status in shoot to the roots. Computational tools discovered miRNA-target interactions by relying on sequence based identification and experimental results supported that miR169 targets member of the NF-YA transcription factor family in eukaryote. NF-Y functions as a trimeric complex composed of three individual subunits, NF-YA, NF-YB and NF-YC (Nardini M. et al., 2013, Cell 152:132-143). NF-Y complex cannot bind to DNA or show transcriptional activation without NF-YA subunit, which has two conserved motifs, NF-YB/NF-YC interaction domain and DNA binding domain. As a transcription factor, NF-YA regulates downstream target gene expression in activation or deactivation manner.

Investigating collaborative regulation of miRNA and transcription factor at the transcriptional and post-transcriptional levels may elucidate their roles in various biological processes (Tran D. H. et al., 2009, Bioinformation 4:371-377). In Arabidopsis thaliana, miR169/NF-YA module is linked with drought stress and associated with carbohydrate metabolism and cell expansion. In maize, the expression of ZmmiR169 and its target genes ZmNF-YAs are conversely responsive to drought, salt, and hormone stresses (Luan et al., 2014, PLoS One. 9, e91369). These literatures indicate that miR169/NF-YA modules regulate tolerance to abiotic stresses in both monocots and dicots. However, the molecular mechanisms of miR169/NF-YA module respond to nitrogen in rice remain relatively unknown.

In this invention, we investigated the physiological function of osa-miRNA169a during the nitrogen response and utilization using transgenic plants. Moreover, we demonstrated that osa-miRNA169a is tightly related to the post-transcriptional regulation of OsNF-YA5. Our results demonstrate that OsNF-YA5/miR169a module regulates NUE depending on the nitrogen status through activation of genes involved in nitrogen use efficiency.

Meanwhile, in Chinese Patent Publication No. 111593058, “Bna-miR169n gene and application thereof in controlling drought resistance of Brassica napus” is disclosed. However, NF-YA5/miR169a module controlling nitrogen utilization efficiency of plant and uses thereof of the present invention have not been described.

### SUMMARY

The present invention is devised under the aforementioned circumstances, and it has been found by the inventors of the present invention that miR169a is acting as a negative regulator of NF-YA5 (Nuclear Factor Y subunit A5) in rice, contributing to reduced nitrogen utilization efficiency. Accordingly, using a gene editing system, miR169a mutants or NF-YA5 mutants with edited miR169a binding site are produced and analyzed for nitrogen utilization efficiency under nitrogen-deficient conditions. As a result, it was found that miR169a mutants or NF-YA5 mutants with edited miR169a binding site exhibit a significantly enhanced nitrogen utilization efficiency compared to non-edited strains, thus the present invention is completed.

To solve the problems described in the above, the present invention provides a method of controlling nitrogen utilization efficiency in plants under nitrogen-deficient conditions, which includes a step of controlling the expression or activity of miR169a consisting of the nucleotide sequence of SEQ ID NO: 37.

The present invention further provides a method of producing a genome-edited rice plant with enhanced nitrogen utilization efficiency under nitrogen-deficient conditions including (a) introducing a guide RNA specific to the target nucleotide sequence of pre-miR169a derived from rice and an endonuclease protein to a rice plant cell to have genome editing; and (b) regenerating a rice plant from the rice plant cell that is obtained after the genome editing.

The present invention further provides a method of producing a genome-edited rice plant with enhanced nitrogen utilization efficiency under nitrogen-deficient conditions including (a) introducing a guide RNA specific to the target nucleotide sequence of NF-YA5 gene derived from rice and an endonuclease protein to a rice plant cell to have genome editing; and (b) regenerating a rice plant from the rice plant cell that is obtained after the genome editing.

The present invention still further provides a genome-edited rice plant with enhanced nitrogen utilization efficiency under nitrogen-deficient conditions which is produced by the aforementioned methods, and a seed thereof.

According to the present invention, it is found to be possible to enhance the nitrogen utilization efficiency in plants under nitrogen-deficient conditions through genetic editing of the NF-YA5/miR169a module. Therefore, the methods of the present invention are expected to be useful in the development of environmentally friendly and cost-effective plants that can reduce the consumption of nitrogen fertilizers while maintaining crop yields.

### BRIEF DESCRIPTION OF THE DRAWINGS

FIGS. 1A to 1D show the results of osa-MIR169a expression under N-deficient conditions and gene expression in osa-MIR169a transgenic plants. FIG. 1A shows the results of OsNF-YA5 and osa-MIR169a in expression in the roots of 4-week-old non-transgenic (NT) plants. Transcript levels were measured by analyzing RNA-seq data. FIG. 1B shows the expression of different miR169 isoforms in roots under modified N media conditions. Rice seedlings were grown on media harboring different concentrations of a N source (KNO₃) for two weeks. Different letters indicate significant differences at P<0.05 (ANOVA followed by Tukey's honestly significant difference). OsNF-YA5 and OsNRT1.1A transcript levels in osa-MIR169a transgenic plants grown under N-sufficient or deficient conditions are shown in FIGS. 1C and 1D, respectively. Total RNA was extracted and transcript levels were measured by RT-qPCR analysis. Transcript levels were normalized to OsUbi1 expression. Values are the means±SD of three biological samples (n=3-5).

FIGS. 2A to 2F show the results of osa-MIR169a expression under N-deficient conditions and gene expression in osa-MIR169a transgenic plants. FIGS. 2A and 2B show the morphology of osa-MIR169a transgenic lines grown in paddy fields for 3 months with different amounts of N fertilizer applied. Applied amount of N fertilizer was 0 kg/are (0% N), 180 g/are (20% N), and 900 g/are (100% N). Scale bar=10 cm. Plant height and fresh weight of osa-MIR169a transgenic plants (3-month-old) in the paddy field are shown in FIGS. 2C and 2D, respectively. FIGS. 2E and 2F show the measurement of agronomic traits, total seed number and total seed weight, in field-grown osa-MIR169a transgenic plants. Values are the means±SD of samples (n>20). Asterisks indicate a statistically significant difference compared with the non-transgenic (NT) control (*P<0.05, ANOVA).

FIGS. 3A to 3D show the results of N assimilating gene expression and amino acid content analysis of osa-MIR169a transgenic plants. FIGS. 3A and 3B show the expression of genes for N uptake and assimilation in osa-MIR169a transgenic plants under N deficient hydroponic culture (2-week-old plants grown under normal N conditions were transferred to N deficient conditions for a week). OsGOGAT1 and OsGS1.1 expression was analyzed by RT-qPCR analysis in shoot and root of OsNF-YA5 transgenic plants, and their results are shown in FIGS. 3A and 3B, respectively. Amino acid content in OsNF-YA5 transgenic plants were grown under N-deficient hydroponic culture. FIGS. 3C and 3D show the total free amino acid and four amino acids involved in nitrogen assimilation in the shoot and root of transgenic plants, respectively. NT, non-transgenic; 169a G #7, GOS23:osa-MIR169a #7; 169a R #7, RCc3:osa-MIR169a #7; 169a KO #2, osa-MIR169a mutant #2. The data represent the mean values±standard deviation (SD) of three sample pool (n=3, each pool with 5 plants). Different letters indicate significant differences between NT and transgenic plants at P<0.05 (ANOVA followed by Tukey's honestly significant difference) (left panel). Asterisks indicate a statistically significant difference compared with NT (*P<0.05, **P<0.01; ANOVA).

FIGS. 4A to 4F show Identification and mutation of osa-MIR169a target site in OsNF-YA5 transcript. FIG. 4A shows schematic diagram and sequence of osa-MIR169a target site in OsNF-YA5 transcript. gRNA for osa-MIR169a target mutant was represented as green color. FIG. 4B shows the PCR product of RLM-RACE and PPM-RACE for osa-MIR169a on OsNF-YA5. M, marker; 1, RLM-RACE product; 2, PPM-RACE product. FIG. 4C shows the comparison of genomic DNA sequence between NT and osa-miR169a target mutant (UTR-M). Plant height and fresh weight of NT and osa-MIR169a target mutant (6-week-old) in the liquid culture are shown in FIGS. 4D and 4E, respectively. FIG. 4F shows the OsNF-YA5 transcript levels in osa-MIR169a target mutant grown under N-sufficient or deficient conditions. Total RNA was extracted and transcript levels were measured by RT-qPCR analysis. Transcript levels were normalized to OsUbi1 expression. Values are the means±SD of three biological samples (n=3-5). Values are the means±SD of samples (n>20). Asterisks indicate a statistically significant difference compared with the non-transgenic (NT) control (*P<0.05, **P<0.01; ANOVA).

FIGS. 5A to 5G show analysis of osa-MIR69a transgenic rice. The expression level of pre-osa-miR169a in the root of RCc3::osa-miR169a and in the leaf of GOS2::osa-miR169a transgenic plants (4-week-old) are shown in FIGS. 5A and 5B, respectively. Total RNA was extracted from rice seedlings, and pre-miR169a transcript level was measured by RT-qPCR analysis. FIG. 5C shows the comparison of genomic DNA sequence between NT and osa-miR169a mutant. FIG. 5D shows the gRNA location for gene editing by CRISPR/Cas9. Bold letters, mature miRNA; Box, gRNA; Underline, PAM. FIG. 5E shows the expression level of pre-osa-miR169a in the root of osa-miR169a mutant. FIG. 5F shows miRNA targets prediction results of psRNATarget analysis. FIG. 5G shows the expression level of NF-YAs in the seedling of GOS2::osa-miR169a (169aOE #4). Transcript levels were normalized to OsUbi1 expression. Values are the means±SD of three biological samples (n=3-5). Asterisks indicate a statistically significant difference compared with NT (*P<0.05, **P<0.01; two-tailed t-test).

### DETAILED DESCRIPTION

To achieve the object of the present invention, the present invention provides a method of controlling nitrogen utilization efficiency in plants under nitrogen-deficient conditions, which includes a step of controlling the expression or activity of miR169a consisting of the nucleotide sequence of SEQ ID NO: 37.

In the method of controlling the nitrogen utilization efficiency according to the present invention, it is preferable that the control of miR169a expression is achieved by knock-out of miR169a using a gene editing system, thereby enhancing the nitrogen utilization efficiency in plants under nitrogen-deficient conditions. However, it is not limited to this method, and any conventional method in the pertinent art that inhibits gene expression may also be possibly employed.

Furthermore, in the method of controlling nitrogen utilization efficiency according to the present invention, the control of miR169a activity can be achieved by knock-out of the target gene of miR169a using a gene editing system, thereby inhibiting the activity of miR169a and enhancing the nitrogen utilization efficiency in plants under nitrogen-deficient conditions. However, it is not limited thereto.

In one implementation of the present invention, the target gene of miR169a may preferably be NF-YA5 (Nuclear Factor Y subunit A5), but it is not limited to NF-YA5. Plants with NF-YA5 gene overexpression are characterized in that they exhibit enhanced nitrogen utilization efficiency under nitrogen-deficient conditions compared to wild-type plants, and miR169a acts as a negative regulator of NF-YA5. In the present invention, the NF-YA5 gene may preferably consist of genomic DNA sequence that includes the binding site for miR169a and may consist of the nucleotide sequence of SEQ ID NO: 40, but it is not limited to this specific sequence.

The present invention further provides a method of producing a genome-edited rice plant with enhanced nitrogen utilization efficiency under nitrogen-deficient conditions including (a) introducing a guide RNA specific to the target nucleotide sequence of pre-miR169a derived from rice and an endonuclease protein to a rice plant cell to have genome editing; and (b) regenerating a rice plant from the rice plant cell that is obtained after the genome editing.

In the method of producing a genome-edited rice plant according to one embodiment of the present invention, the pre-miR169a derived from rice may include, as a precursor sequence of miR169a, the mature miR169a sequence, and it may consists of the nucleotide sequence of SEQ ID NO: 38. In the present invention, the target nucleotide sequence of the pre-miR169a derived from rice may be the nucleotide sequence of SEQ ID NO: 39, but it is not limited to this specific sequence.

The present invention further provides a method of producing a genome-edited rice plant with enhanced nitrogen utilization efficiency under nitrogen-deficient conditions including (a) introducing a guide RNA specific to the target nucleotide sequence in NF-YA5 gene derived from rice and an endonuclease protein to a rice plant cell to have genome editing; and (b) regenerating a rice plant from the rice plant cell that is obtained after the genome editing.

In the method of producing a genome-edited rice plant according to one embodiment of the present invention, the target nucleotide sequence of NF-YA5 gene derived from rice may be the nucleotide sequence of SEQ ID NO: 41, but it is not limited to this specific sequence. Furthermore, the target nucleotide sequence of NF-YA5 gene derived from rice consisting of the nucleotide sequence of SEQ ID NO: 41 is a binding site for miR169a.

As described herein, the term “genome/gene editing” means a technique for introducing a target-oriented mutation to a genome nucleotide sequence of a plant or an animal cell including human cell. Specifically, it indicates a technique for knock-out or knock-in of a specific gene by deletion, insertion, or substitution of one more nucleic acid molecules by DNA cutting, or a technique for introducing a mutation even to a non-coding DNA sequence which does not produce any protein. According to the purpose of the present invention, the genome editing may be an introduction of a mutation to a plant by using an endonuclease, for example, Cas9 (CRISPR associated protein 9) protein, and a guide RNA. Furthermore, the term “gene editing” may be interchangeably used with “gene engineering”.

Furthermore, the term “target gene” described herein means part of DNA which is present in the genome of a plant and to be edited according to the present invention. Type of the target gene is not limited, and it may include a coding region and also a non-coding region. Depending on the purpose, a person who is skilled in the pertinent art may select the target gene according to the mutation of a plant that is desired to be produced by genome editing.

Furthermore, the term “guide RNA” described herein means a ribonucleic acid which includes RNA specific to a target DNA in nucleotide sequence encoding the target gene and, according to complementary binding between the whole or partial sequence of the guide RNA and the nucleotide sequence of target DNA, the guide RNA plays the role of guiding an endonuclease to the target DNA nucleotide sequence. The guide RNA represents two types of RNA, i.e., a dual RNA having crRNA (CRISPR RNA) and tracrRNA (trans-activating crRNA) as a constitutional element; or a single chain guide RNA (sgRNA) including the first site which includes a sequence that is fully or partially complementary to the nucleotide sequence in the target gene and the second site which includes a sequence interacting with RNA-guide nuclease, but those in the form in which the RNA-guide nuclease is active for the target nucleotide sequence are also within the scope of the invention without any limitation. Considering the type of an endonuclease to be used together, microorganisms from which the endonuclease is derived, or the like, the guide RNA may be suitably selected according to the technique that is well known in the pertinent art.

Furthermore, the guide RNA may be those transcribed from a plasmid template, those obtained by in vitro transcription (e.g., oligonucleotide double strand), or those obtained by synthesis, or the like, but it is not limited thereto.

Furthermore, with regard to the method of producing a genome-edited rice plant according to the present invention, the endonuclease protein may be one or more selected from a group consisting of Cas9, Cpf1 (CRISPR from Prevotella and Francisella 1), TALEN (Transcription activator-like effector nuclease), ZFN (Zinc Finger Nuclease), and a functional analog thereof. It may be preferably Cas9 protein, but it is not limited thereto.

Furthermore, the Cas9 protein may be one or more selected from a group consisting of Cas9 protein derived from Streptococcus pyogenes, Cas9 protein derived from Campylobacter jejuni, Cas9 protein derived from S. thermophilus, Cas9 protein derived from S. aureus, Cas9 protein derived from Neisseria meningitides, Cas9 protein derived from Pasteurella multocida, Cas9 protein derived from Francisella novicida, and the like, but it is not limited thereto. Information of Cas9 protein or gene of Cas9 protein can be obtained from a known database like GenBank of NCBI (National Center for Biotechnology Information).

Cas9 protein is an RNA-guided DNA endonuclease enzyme which induces breakage of a double stranded DNA. In order for Cas9 protein to cause DNA breakage after precise binding to a target nucleotide sequence, a short nucleotide sequence consisting of three nucleotides, which is known as PAM (Protospacer Adjacent Motif), should be present next to the target nucleotide sequence, and Cas9 protein causes the breakage by assuming the position between the 3rd and the 4th base pairs from the PAM sequence (NGG).

In the present invention, the guide RNA and endonuclease protein may function as RNA gene scissors (RNA-Guided Engineered Nuclease, RGEN) by forming a ribonucleic acid-protein (i.e., ribonucleoprotein) complex.

The CRISPR/Cas9 system employed in the present invention is a method of gene editing based on NHEJ (non-homologous end joining) mechanism in which insertion-deletion (InDel) mutation resulting from incomplete repair, which is induced during a process of DNA repair, is caused by introducing breakage of a double strand at specific site of a specific gene to be edited.

With regard to the method of producing a genome-edited rice plant according to the present invention, introducing guide RNA and endonuclease protein to a rice plant cell of the aforementioned step (a) may involve use of: a complex (i.e., ribinucleoprotein) of a guide RNA specific to the target nucleotide sequence of pre-miR169a or NF-YA5 gene derived from rice and an endonuclease protein; or a recombinant vector having a DNA encoding a guide RNA specific to the target nucleotide sequence of pre-miR169a or NF-YA5 gene derived from rice and a nucleic acid sequence encoding an endonuclease protein, but it is not limited thereto.

In the present specification, the term “recombinant” indicates a cell which replicates a heterogeneous nucleotide or expresses said nucleotide, or a peptide, a heterogeneous peptide, or a protein encoded by a heterogeneous nucleotide. Recombinant cell can express a gene or a gene fragment in the form of a sense or antisense, which are not found in natural state of cell. In addition, a recombinant cell can express a gene that is found in natural state, provided that said gene is modified and re-introduced into the cell by an artificial means.

The term “vector” is used herein to refer DNA fragment (s) and nucleotide molecules that are delivered to a cell. Vector can replicate DNA and be independently reproduced in a host cell. The terms “delivery system” and “vector” are often interchangeably used.

The vector of the present invention can be constructed as a vector which is typically used for cloning or expression. In addition, the vector of the present invention can be constructed by having a prokaryotic cell or a eukaryotic cell as a host. For example, when the vector of the present invention is an expression vector and a prokaryotic cell is employed as a host, a strong promoter for the initiation of transcription (e.g., pLλ promoter, trp promoter, lac promoter, T7 promoter, tac promoter and the like), and a ribosome binding site for the initiation of translation and a termination sequence for transcription/translation are generally included. When E. coli is employed as a host cell, a promoter and an operator region relating to the biosynthetic pathway of tryptophan in E. coli, and left-side promoter of phage λ (i.e., pLλ promoter) can be used as a regulation site.

For the recombinant vector according to the present invention, the promoter may be any of CaMV 35S promoter, actin promoter, ubiquitin promoter, pEMU promoter, MAS promoter, and histone promoter, but not limited thereto.

In the present specification, the term “promoter” means a DNA molecule to which RNA polymerase binds in order to initiate its transcription, and it corresponds to a DNA region upstream of a structural gene. The term “plant promoter” indicates a promoter which can initiate transcription in a plant cell. The term “constitutive promoter” indicates a promoter which is active in most of environmental conditions and development states or cell differentiation states. Since a transformant can be selected with various mechanisms at various stages, the constitutive promoter can be preferable for the present invention. Therefore, a possibility for choosing the constitutive promoter is not limited herein.

The recombinant vector of the present invention can be constructed according to a method which is well known to a skilled person in the art. The method includes an in vitro recombinant DNA technique, a DNA synthesis technique, and an in vivo recombinant technique. For inducing mRNA synthesis, the DNA sequence can be effectively linked to a suitable promoter present in the expression vector. In addition, the expression vector may include a ribosome binding site as a translation initiation site and a transcription terminator.

Preferred example of the recombinant vector of the present invention is Ti-plasmid vector which can transfer a part of itself, i.e., so called T-region, to a plant cell when the vector is present in an appropriate host such as Agrobacterium tumefaciens. Other types of Ti-plasmid vector (see, EP 0 116 718 B1) are currently used for transferring a hybrid DNA sequence to protoplasts that can produce a new plant by appropriately inserting a plant cell or hybrid DNA to a genome of a plant. Especially preferred form of Ti-plasmid vector is a so-called binary vector which has been disclosed in EP 0 120 516 B1 and U.S. Pat. No. 4,940,838. Other vector that can be used for introducing the DNA of the present invention to a host plant can be selected from a double-stranded plant virus (e.g., CaMV), a single-stranded virus, and a viral vector which can be originated from Gemini virus, etc., for example a non-complete plant viral vector. Use of said vector can be advantageous especially when a host plant cannot be easily transformed.

The recombinant vector may include at least one selective marker. Said selective marker is a nucleotide sequence having a property of being selected by a common chemical method. Examples include all genes that are useful for distinguishing transformed cells from non-transgenic cells. Specific examples thereof include a gene resistant to herbicide such as glyphosate and phosphinotricine, and a gene resistant to antibiotics such as kanamycin, G418, bleomycin, hygromycin, chloramphenicol, and aadA gene, but not limited thereto.

For the recombinant vector of the present invention, any conventional terminator can be used. Examples include nopaline synthase (NOS), rice α-amylase RAmy1 A terminator, a phaseolin terminator, a terminator for octopine gene of Agrobacterium tumefaciens, rmB1/B2 of Escherichia coli or the like, but are not limited thereto.

Any plant cell can be employed as the “plant cell” that is used for transformation of a plant. The plant cell may be cultured cells, cultured tissues, cultured organs, or whole plant, preferably cultured cells, cultured tissues, or cultured organs, and more preferably cultured cells in any form. The “plant tissue” may be either differentiated or undifferentiated tissues of a plant, and examples thereof include, although not limited thereto, root, stem, leaf, pollen, seed, tumor tissue, and cells in various forms that are used for culture like single cell, protoplast, shoot, and callus tissue. The plant tissue can be either in planta, or in a state of organ culture, tissue culture, or cell culture.

The present invention still further provides a genome-edited rice plant with enhanced nitrogen utilization efficiency under nitrogen-deficient conditions which is produced by the aforementioned methods of producing genome-edited rice plant, and a seed thereof.

The genome-edited rice plant according to the present invention is characterized in that it has enhanced nitrogen utilization efficiency under nitrogen-deficient conditions as rice-derived pre-miR169a or NF-YA5 gene is knocked out.

Hereinbelow, the present invention is explained in greater detail in view of the Examples. However, it is evident that the following Examples are given only for exemplification of the present invention and by no means the present invention is limited to the following Examples.

### Materials and Methods

### Plant Growth Conditions

Rice (Oryza sativa cv. Dongjin) seeds were sown on a Murashige-Skoog (MS) solid medium and incubated in the dark for 4 days at 28° C. Seedlings were then transferred to a growth chamber with a light and dark cycle of 16 h light/8 h dark, a light intensity of 200 μmol m⁻² s⁻¹, and relative humidity of 70%. Two-week-old seedlings were used for gene expression analysis. To examine the nitrogen concentration-dependent response of osa-miR169s, rice seeds were germinated on solid MS media without nitrogen (Caisson Labs, USA) supplemented with various concentrations of KNO₃ or NH₄NO₃. Seedlings were harvested two weeks after germination for RNA extraction. We prepared liquid culture solutions with nutrient compositions as previously described (Redillas et al., 2019, Plant Biotechnol J 17:1289-1301), and the nitrogen-deficient solution was prepared as in the same paper except for the omission of the N source (NH₄NO₃). For the liquid culturing of rice, we prepared the culture solution as described in our previous report.

### Plasmid Construction and Rice Transformation

To generate overexpression plants, the genomic region surrounding the pri-microRNA sequence of osa-miR169a were amplified from rice (O. sativa L. ssp. japonica cv. Nipponbare) total RNA using the Reverse Transcription System (Promega, USA) and PrimeSTAR HS DNA polymerase (TAKARA, Japan). The PCR-amplified osa-miR169a genomic sequence were cloned into the rice transformation vector p700 carrying the GOS2 promoter for constitutive overexpression or the RCc3 promoter for root-preferential overexpression using Gateway cloning system (Thermo Fisher, USA) (Jeong et al., 2010, Plant Physiol. 153:185-197). For CRISPR/Cas9-mediated mutagenesis of the OsNF-YA5 3′ UTR, guide RNA (gRNA) targeting the miRNA target binding site were cloned into a CRISPR/Cas9 expression vector carrying a rice codon-optimized Streptococcus pyrogenes Cas9 (rCRISPR/Cas9) using the XhoI and BamHI restriction enzyme sites. For CRISPR/Cas9-mediated mutagenesis of the osa-miR169a, guide RNA (gRNA) targeting the miRNA target binding site were cloned into a CRISPR/Cas9 expression vector carrying a rice codon-optimized Streptococcus pyrogenes Cas9 (rCRISPR/Cas9) using the XhoI and BamHI restriction enzyme sites. To minimize the possibility of off-target effects caused by the CRISPR/Cas9 mediated mutagenesis, we performed a computational analysis to select unique target site-specific gRNAs on the pri-miRNA sequence of osa-miR169s using CRISPR-P v2.0 and CRISPRdirect. Primers used for vector construction are listed in Table 1.

TABLE 1
List of primer sequences used in this invention

| PCR primers | 5′ to 3′ | |
|---|---|---|
| qRT-PCR | Foward | Reverse |
| osNF-YA5 | CGCCATTGCAGGAGTACCAA (SEQ ID NO: 1) | TGCAGAAGTTGGTGCAAACC (SEQ ID |
| | | NO: 2) |
| osNF-YA1 | CTGACCGTAGCCAACCCTTT (SEQ ID NO: 3) | CTCGGGAGGATGACATCACG (SEQ ID |
| | | NO: 4) |
| osNF-YA2 | TGACAGCAGTCTCAACGGAC (SEQ ID NO: 5) | GGAGGAAAGGCAGCTTCTGT (SEQ ID |
| | | NO: 6) |
| osNF-YA6 | GAAATCGCGAGCGTCTCAAC (SEQ ID NO: 7) | CTCATCATGGAAACGCGCTG (SEQ ID |
| | | NO: 8) |
| osNF-YA10 | CTGGGCCTTGGTCAATCTGT (SEQ ID NO: 9) | TTGGTGCATCAGCTGGCATA (SEQ ID |
| | | NO: 10) |
| osNF-YA11 | ACCACCACAGACAGCCAAAA (SEQ ID NO: 11) | GTTTGATCTCCGATTGCGCC (SEQ ID |
| | | NO: 12) |
| Osmi-R169a | TTAAGCAGCTAGCCGGGAAT (SEQ ID NO: 13) | GCCAAGAACAACTTGCCAT (SEQ ID |
| | | NO: 14) |
| Osmi-R169h | TGGTCCTGAAGAGTTGCAGA (SEQ ID NO: 15) | AAGGACACAGGCAAGTCATC (SEQ ID |
| | | NO: 16) |
| Osmi-R169i | GAGATGGAAGAGAGCAAGGC (SEQ ID NO: 17) | CTCTACACAAGGACACAGGC (SEQ ID |
| | | NO: 18) |
| OsNRT1.1A | GTGACTCGAGGTTGGTGCAT (SEQ ID NO: 19) | TGATGAAGCCGTGGTGTTCT (SEQ ID |
| | | NO: 20) |
| [symbol] -F-q | ATGGAGCTGCTGCTGTTCTA (SEQ ID NO: 21) | TTCTTCCATGCTGCTCTACC (SEQ ID |
| | | NO: 22) |
| PLM, PPM RACE | | |
| 5′ adapter | CGACUGGAGCACAGGACACUGACAUGGACUGAAGGAGUAGAAA (SEQ ID NO: 23) | |
| Oligo(dT) adapter | ATTCTAGAGGCCGAGGCGGCCGACATG-d(T) [symbol] (SEQ ID NO: 24) | |
| Gene Racer 5′ | AGGACACTGACATGGACTGAAGGAGTAG (SEQ ID NO: 25) | |
| Gene Racer 3′ | ATTCTAGAGGCCGAGGCGGCCGACATG (SEQ ID NO: 26) | |
| GSP F | GCGTCGACCTCCTCAAGGTGTGA (SEQ ID NO: 27) | |
| GSP R | GCAACTGAGAAGCAGTTTGCATCAGTTGC (SEQ ID NO: 28) | |
| CRISPR/Cas9 | | |
| OsU3pro_Hind3_F | CCCAAGCTTAAGGAATCTTTAAACATACGA (SEQ ID NO: 29) | |
| gRNA-Ter | TGCTCTAGAAAAACAAAAAAGCACCGACTCGGTGC (SEQ ID NO: 30) | |
| (XbaI)_R | | |
| OsU3_3′UTR_sgRNA_ | CGCCGGTGGCAATTCATCCTGTTTTAGAGCTAGAAATAGC (SEQ ID NO: 31) | |
| F | | |
| [symbol] _NP- | AGGATGAATTGCCACCGGCGGCCACGGATCATCTGCA (SEQ ID NO: 32) | |
| FYA5_3′UTR_ | | |
| sgRNA_R | | |
| OsU3_miR169a_ | CCCTGCTCCTCATGTAAGGCGTTTTAGAGCTAGAAATAGC (SEQ ID NO: 33) | |
| sgRNA_P | | |
| [symbol] _miR169a_R | GCCTTACATGAGGAGCAGGGCCACGGATCATCTGCA (SEQ ID NO: 34) | |
| Cloning | Foward | Reverse |
| pENTR_miR169a | CACCCTGAACTCATCATTCTTCTCC (SEQ | ATAGAGGAGGTAGTACTTATC (SEQ |
| | ID NO: 35) | ID NO: 36) |

[symbol]

indicates data missing or illegible when filed

The constructs were transformed into rice plants (O. sativa cv. Dongjin) by Agrobacterium tumefaciens (LBA4404)-mediated co-cultivation, as previously described. The selected transgenic plants were self-fertilized, and homozygous transgenic lines were selected from T2 generations by analyzing the segregation ratio on MS media containing 4 mg/L phosphinothricin (Duchefa, Netherlands). Three independent single-copy insertion homozygous plants were selected and propagated in a rice paddy field at Kyungpook National University, Gunwi (128:34E/36:15N), Korea for further propagation.

### RNA Isolation and RT-qPCR Analysis

Total RNA samples were extracted from rice leaf or root tissues using a Hybrid-R RNA purification kit (GeneAll Biotechnology, Korea) according to the manufacturer's instructions. To synthesize the first-strand complementary DNA (cDNA), 2 μg of total RNA were reverse-transcribed using RevertAid M-MuLV Reverse Transcriptase (Thermo Scientific, USA). RT-qPCR analysis was performed using the Real-Time PCR smart mix (SolGent, Korea) and EvaGreen (SolGent, Korea) in an AriaMx real-time PCR System (Agilent, USA). The PCR reactions were performed by initial denaturation at 95° C. for 10 min, followed by 40 cycles of 95° C. for 20 s, 60° C. for 20 s, and 72° C. for 30 s. Rice Ubiquitin1 (Os06g0681400) was used as an internal control for normalization. Three technical replicates were analyzed for quantitative experiments. Primers used for RT-qPCR are listed in Table 1.

### RLM-RACE and PPM-RACE Analysis

To validate miRNA target site, we performed RLM-RACE and PPM-RACE methods. The detailed procedures of these methods are described previous report (Wang and Fang, 2015, Methods mol. biol. 1296:175-186). And we followed their protocol. Primers for RACE analysis was listed in Table 1.

### Agronomic Trait Analysis of Rice Plants Grown in a Paddy Field

The agronomic traits of the NT plants and two independent T4 homozygous transgenic lines planted in a paddy field at the Kyungpook National University, Gunwi (128:34E/36:15N), Korea in 2020 were analyzed. Three separated paddy fields were used, and different amounts of N fertilizer (0% N, 0 g/are; 20% N, 180 g/are; 100% N, 900 g/are) were applied to each field, while the same amount of other nutrients, including P and K, were supplied to all the fields. The experiment included three replicates where three different plots were planted in a randomized design. The yield components of 30 plants per line from the three different plots in each field were measured and analyzed.

### Amino Acid Analysis

Amino acid quantification was performed by the National Instrumentation Center for Environmental Management (NICEM) in Seoul National University, Korea. Analysis was done using a HPLC Ultimate 3000 equipped with a VD Spher 100 C18-E column (4.6 mm×150 mm, 3.5 μm/VDS, Optilab, Germany) and FL 1260 FLD detector (Agilent Technologies, USA) according to the manufacturer's manual.

### Statistical Analysis

All data are represented as the mean value±standard deviation. Each data value was separately compared to the control value to determine significantly differences using a t-test (*P<0.05, **P<0.01) or ANOVA and Tukey's test. Data were analyzed using the Microsoft Excel or IBM SPSS software.

### Example 1. Osa-miR169a Negatively Regulates Transcript Levels of OsNF-YA5 and Nitrogen Use Efficiency

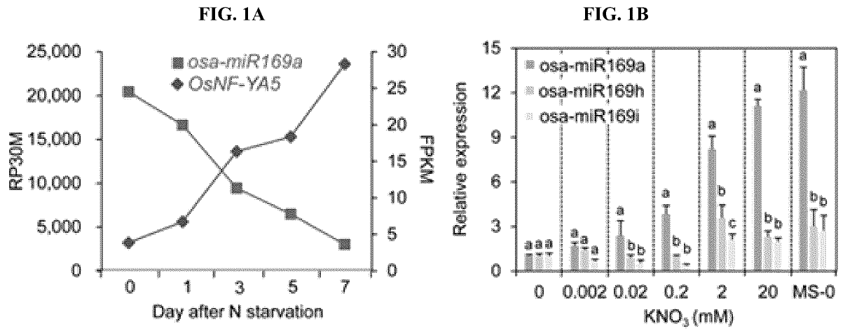

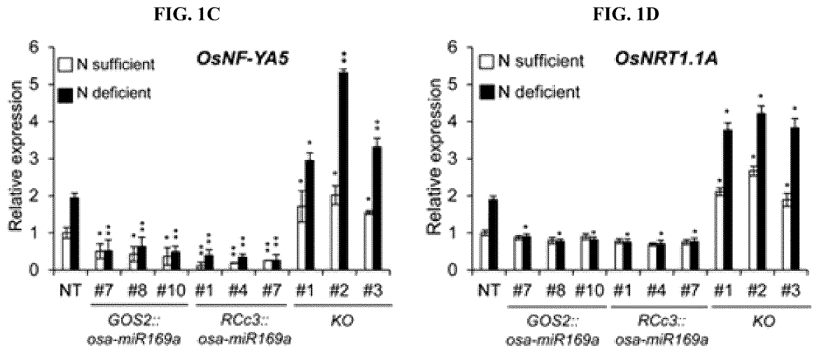

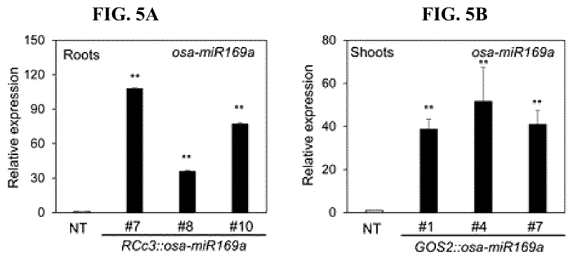

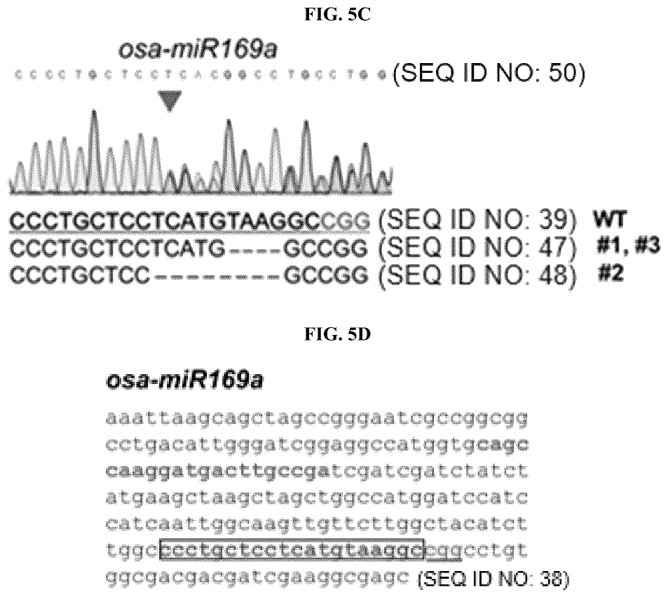

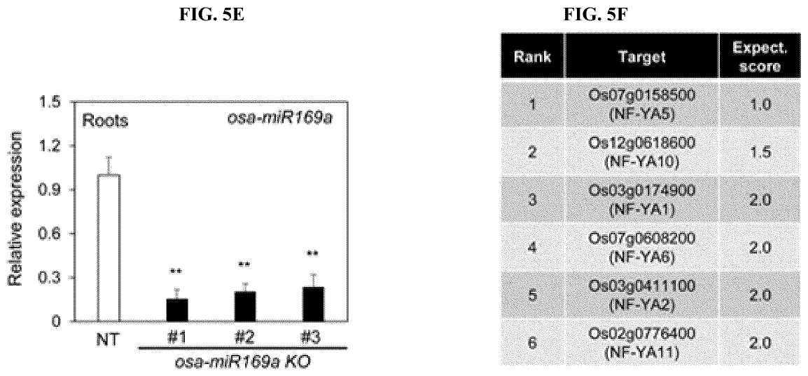

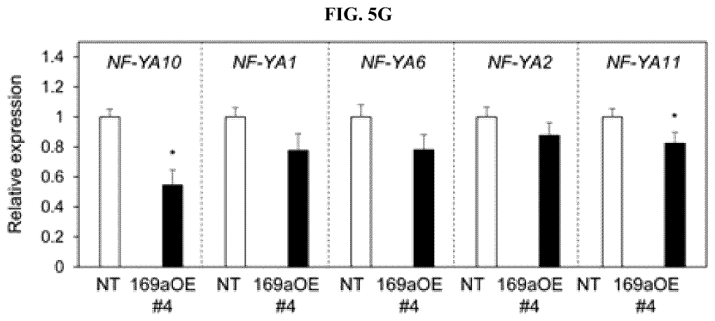

It was reported that miR169 directly targets NF-YA family transcripts MA. thaliana. Among several miR169 isoforms in rice, we observed that osa-miR169a expression sharply decreased during nitrogen (N) starvation conditions (FIG. 1A). To address the relationship between osa-miR169a and N concentration, we measured precursor miRNA169a (osa-pre-miR169a) expression levels in plants grown in media with different N concentrations (FIG. 1B). osa-pre-miR169a transcript levels decreased under low N conditions and significantly increased under high N conditions, suggesting that osa-miR169a plays a role in N signaling. To identify the osa-miR169a target, we used a miRNA targets prediction tool, psRNATarget (http://plantgm.noble.org/psRNATarget/), which highlighted OsNF-YA5 as the most likely osa-miR169a target (FIG. 5F). Moreover, we checked the expression of other putative targets of osa-miR169a and confirmed that OsNF-YA5 is the major target of osa-miR169a (FIG. 5G). To confirm the relationship between osa-miR169a and OsNF-YA5, we generated osa-MIR169a OE and KO plants (FIGS. 5A to 5E) and observed that OsNF-YA5 expression was significantly lower in osa-MIR169a OE plants and higher in KO mutants compared to NT plants, suggesting that osa-miR169a negatively regulates OsNF-YA5 transcript levels (FIG. 1C). Similarly, OsNRT1.1A, target gene of OsNF-YA5, transcript levels in osa-miR169a OE plants were lower than in NT plants (FIG. 1D). The results suggest that osa-miR169a post-transcriptionally regulates the OsNF-YA5 transcript level depending on the N status.

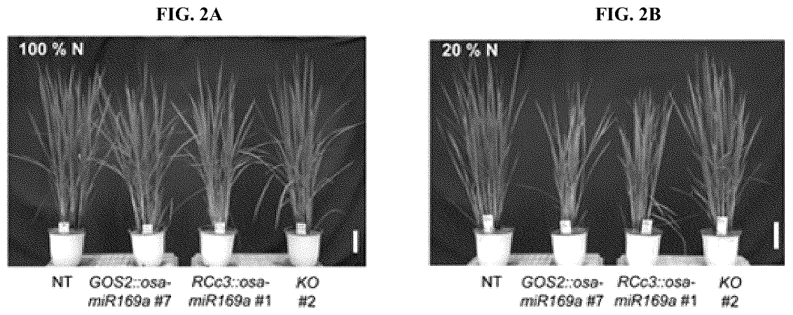

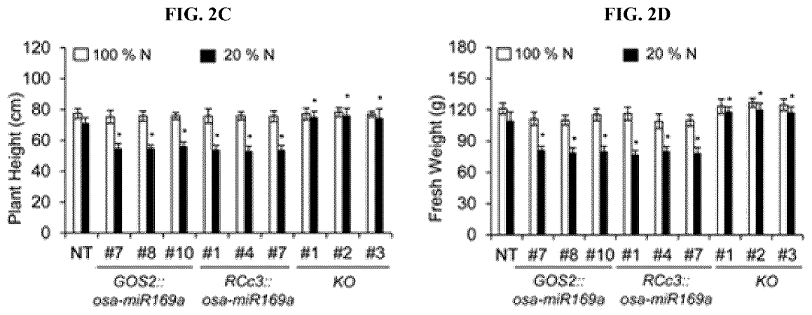

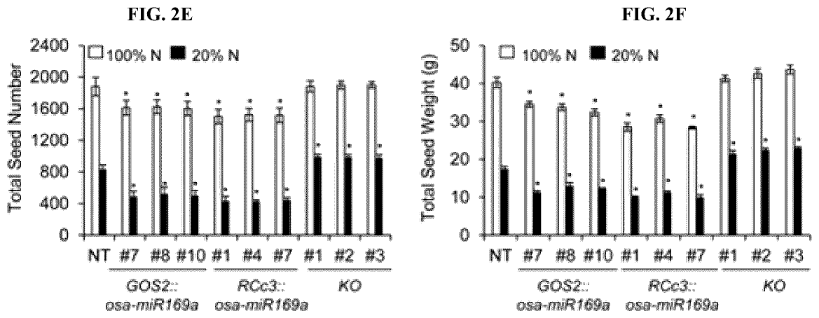

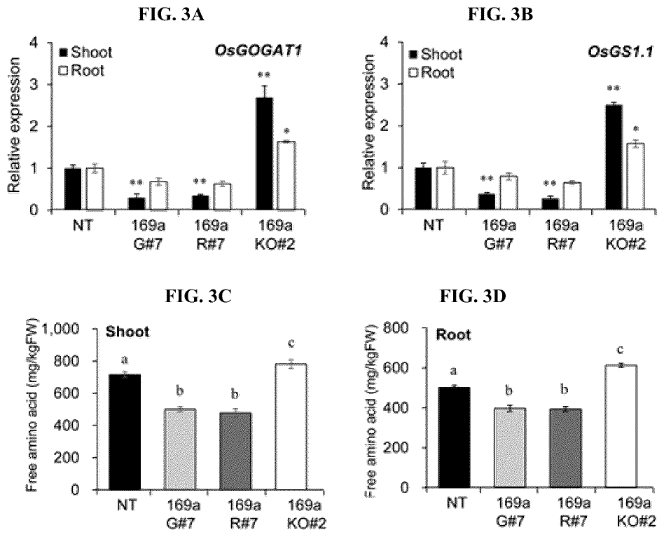

Plant growth reduction of osa-MIR169a OE was significant under N-deficient field conditions, while plant growth of osa-MIR169a KO plants was almost unaffected under N-deficient conditions (FIGS. 2A to 2D). Agronomic traits, including total seed number and seed weight, of osa-MIR169a OE plants decreased significantly, whereas those of osa-MIR169a KO plants increased compared to NT under N-deficient conditions (FIGS. 2E and 2F). In addition, the expression level of N assimilating genes, GOGAT1 and GS1, were significantly down-regulated in osa-MIR169a OE shoots and up-regulated in osa-MIR169a KO shoots compared to NT plants under N-deficient conditions (FIGS. 3A and 3B). Also, free amino acid content was lower in osa-MIR169a OE plants and higher in osa-MIR169a KO plants than that in NT plants (FIGS. 3C and 3D). These results demonstrated that osa-miR169a directly regulates OsNF-YA5 transcript levels and that the OsNF-YA5/osa-miR169a module tightly regulates N utilization, depending on N status.

### Example 2. Mutation of Osa-miR169a Target Site in OsNF-YA5 Improves Nitrogen Use Efficiency

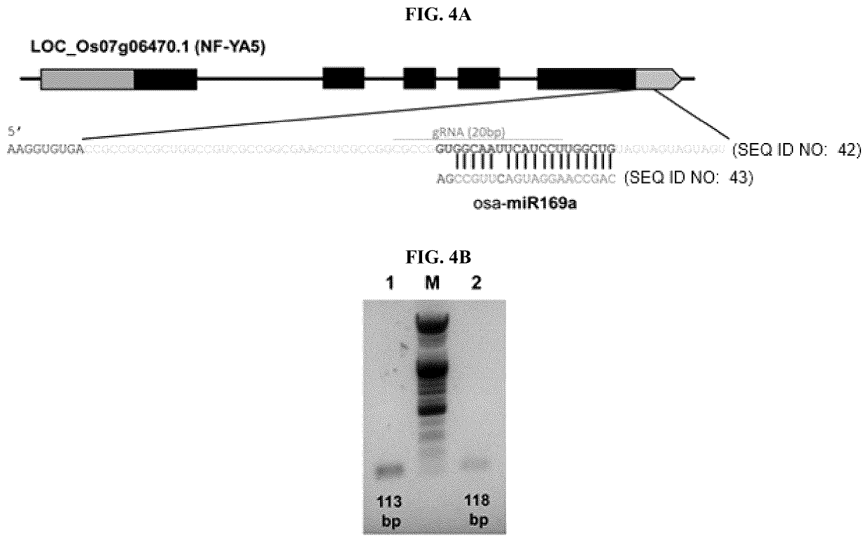

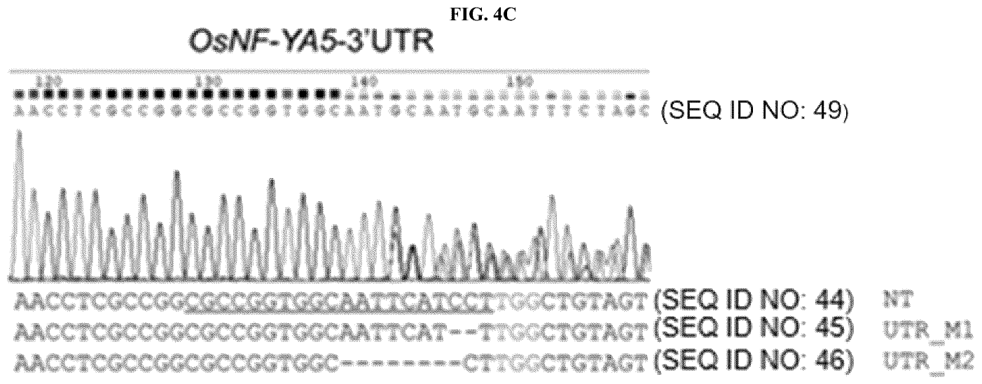

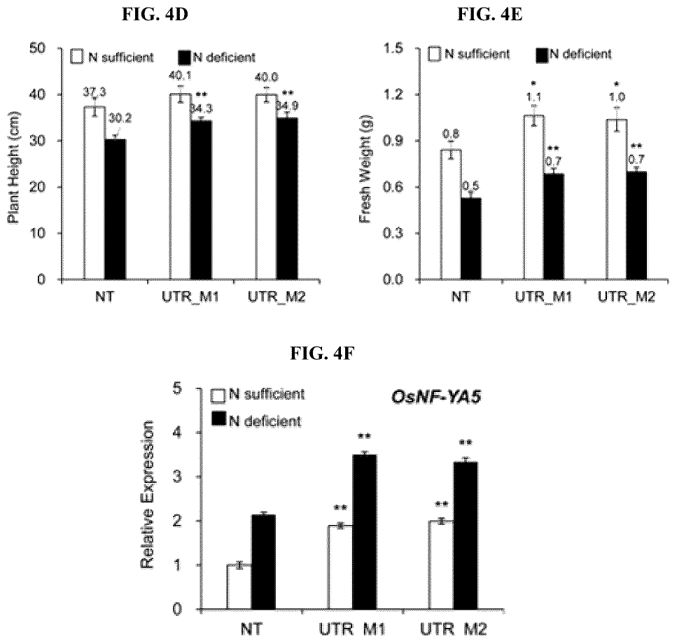

Using miRNA targets prediction tool, psRNATarget, the putative osa-MIR169a binding site in OsNF-YA5 transcript was identified and it is located in 3′ UTR region of OsNF-YA5 transcript (FIG. 4A). To check whether the putative target site was cleavage by osa-MIR169a, we performed RNA Ligase-Mediated (RLM)—Rapid Amplification of cDNA Ends (RACE) and Poly (A) Polymerase-Mediated (PPM)—RACE experiments. The result showed that OsNF-YA5 transcript was truncated in the cleavage site (FIG. 4B). These results demonstrated that miRNA target site of OsNF-YA5 mRNA is located in 3′ UTR and regulates OsNF-YA5 mRNA stability.

To improve the N utilization efficiency by enhancing the stability of OsNF-YA5 transcript, we generated the transgenic plants harboring the mutation of miRNA target site of OsNF-YA5 by CRISPR/Cas9 system. Two different transgenic lines was isolated (UTR_M1 and M2) and confirmed the mutation by PCR sequencing (FIG. 4C). The plant height was not significantly different between NT and mutants under N sufficient conditions; however, the height of mutants was bigger than that of NT under N deficient conditions (FIG. 4D). The fresh weight of mutants was significantly higher than that of NT under both N sufficient and deficient conditions (FIG. 4E). Moreover, OsNF-YA5 expression level was significantly enhanced in the mutants compared to NT plants under both N sufficient and deficient conditions (FIG. 4F). These results demonstrate that OsNF-YA5 transcript was stabilized by the mutation of miRNA target site, which improves plant N utilization and growth.

This invention was made with government support under Cooperative Research Program for Agriculture Science and Technology Development (Project No. PJ01700802 to J.-K.K), awarded by the Rural Development Administration, Republic of Korea.

### REFERENCE TO AN ELECTRONIC SEQUENCE LISTING

A sequence listing electronically submitted on Sep. 15, 2023 as an XML file named 20230915_520623GR11_TU_SEQ.XML, created on Sep. 14, 2023 and having a size of 62,095 bytes, is incorporated herein by reference in its entirety.

What is claimed is:

1. A method of controlling nitrogen utilization efficiency in a plant under a nitrogen-deficient condition, the method comprising:
controlling expression and/or activity of miR169a consisting of the nucleotide sequence of SEQ ID NO: 37.

2. The method according to claim 1, wherein the method comprises the controlling of the expression comprising knocking out miR169a using a gene editing system, thereby increasing the nitrogen utilization efficiency in the plant under the nitrogen-deficient condition.

3. The method according to claim 1, wherein the method comprises the controlling of the activity comprising inhibiting activity of miR169a based on knock-out of a target gene of miR169a using a gene editing system, thereby increasing the nitrogen utilization efficiency in the plant under the nitrogen-deficient condition.

4. The method according to claim 3, wherein the target gene of miR169a is NF-YA5 (Nuclear Factor Y subunit A5).

5. A method of producing a genome-edited rice plant with enhanced nitrogen utilization efficiency under a nitrogen-deficient condition, the method comprising:
introducing a guide RNA specific to a target nucleotide sequence of pre-miR169a derived from rice and an endonuclease protein to a rice plant cell to have genome editing; and
regenerating a rice plant from the rice plant cell that is obtained after the genome editing.

6. The method according to claim 5, wherein the target nucleotide sequence of pre-miR169a derived from rice consists of the nucleotide sequence of SEQ ID NO: 39.

7. A method of producing a genome-edited rice plant with enhanced nitrogen utilization efficiency under a nitrogen-deficient condition, the method comprising:
introducing a guide RNA specific to a target nucleotide sequence of NF-YA5 gene derived from rice and an endonuclease protein to a rice plant cell to have genome editing; and
regenerating a rice plant from the rice plant cell that is obtained after the genome editing.

8. The method according to claim 7, wherein the target nucleotide sequence of NF-YA5 gene derived from rice consists of the nucleotide sequence of SEQ ID NO: 41.

9. The method according to claim 8, wherein the target nucleotide sequence of NF-YA5 gene derived from rice is a binding site for miR169a.

10. A genome-edited rice plant with enhanced nitrogen utilization efficiency under the nitrogen-deficient conditions which is produced by the method of claim 7.

11. A seed of the genome-edited rice plant of claim 10.


SEQUENCE LISTING (50 sequences)

SEQ ID NO: 1 - DNA, 20 bp, synthetic construct
  misc_feature 1..20: note = OsNF-YA5
  source 1..20: mol_type = other DNA
cgccattgcaggagtaccaa

SEQ ID NO: 2 - DNA, 20 bp, synthetic construct
  misc_feature 1..20: note = OsNF-YA5
  source 1..20: mol_type = other DNA
tgcagaagttggtgcaaacc

SEQ ID NO: 3 - DNA, 20 bp, synthetic construct
  misc_feature 1..20: note = OsNF-YA1
  source 1..20: mol_type = other DNA
ctgaccgtagccaacccttt

SEQ ID NO: 4 - DNA, 20 bp, synthetic construct
  misc_feature 1..20: note = OsNF-YA1
  source 1..20: mol_type = other DNA
ctcgggaggatgacatcacg

SEQ ID NO: 5 - DNA, 20 bp, synthetic construct
  misc_feature 1..20: note = OsNF-YA2
  source 1..20: mol_type = other DNA
tgacagcagtctcaacggac

SEQ ID NO: 6 - DNA, 20 bp, synthetic construct
  misc_feature 1..20: note = OsNF-YA2
  source 1..20: mol_type = other DNA
ggaggaaaggcagcttctgt

SEQ ID NO: 7 - DNA, 20 bp, synthetic construct
  misc_feature 1..20: note = OsNF-YA6
  source 1..20: mol_type = other DNA
gaaatcgcgagcgtctcaac

SEQ ID NO: 8 - DNA, 20 bp, synthetic construct
  misc_feature 1..20: note = OsNF-YA6
  source 1..20: mol_type = other DNA
ctcatcatggaaacgcgctg

SEQ ID NO: 9 - DNA, 20 bp, synthetic construct
  misc_feature 1..20: note = OsNF-YA10
  source 1..20: mol_type = other DNA
ctgggccttggtcaatctgt

SEQ ID NO: 10 - DNA, 20 bp, synthetic construct
  misc_feature 1..20: note = OsNF-YA10
  source 1..20: mol_type = other DNA
ttggtgcatcagctggcata

SEQ ID NO: 11 - DNA, 20 bp, synthetic construct
  misc_feature 1..20: note = OsNF-YA11
  source 1..20: mol_type = other DNA
accaccacagacagccaaaa

SEQ ID NO: 12 - DNA, 20 bp, synthetic construct
  misc_feature 1..20: note = OsNF-YA11
  source 1..20: mol_type = other DNA
gtttgatctccgattgcgcc

SEQ ID NO: 13 - DNA, 20 bp, synthetic construct
  misc_feature 1..20: note = OsmiR169a
  source 1..20: mol_type = other DNA
ttaagcagctagccgggaat

SEQ ID NO: 14 - DNA, 20 bp, synthetic construct
  misc_feature 1..20: note = OsmiR169a
  source 1..20: mol_type = other DNA
gccaagaacaacttgccaat

SEQ ID NO: 15 - DNA, 20 bp, synthetic construct
  misc_feature 1..20: note = OsmiR169h
  source 1..20: mol_type = other DNA
tggtcctgaagagttgcaga

SEQ ID NO: 16 - DNA, 20 bp, synthetic construct
  misc_feature 1..20: note = OsmiR169h
  source 1..20: mol_type = other DNA
aaggacacaggcaagtcatc

SEQ ID NO: 17 - DNA, 20 bp, synthetic construct
  misc_feature 1..20: note = OsmiR169i
  source 1..20: mol_type = other DNA
gagatggaagagagcaaggc

SEQ ID NO: 18 - DNA, 20 bp, synthetic construct
  misc_feature 1..20: note = OsmiR169i
  source 1..20: mol_type = other DNA
ctctacacaaggacacaggc

SEQ ID NO: 19 - DNA, 20 bp, synthetic construct
  misc_feature 1..20: note = OsNRT1.1A
  source 1..20: mol_type = other DNA
gtgactcgaggttggtgcat

SEQ ID NO: 20 - DNA, 20 bp, synthetic construct
  misc_feature 1..20: note = OsNRT1.1A
  source 1..20: mol_type = other DNA
tgatgaagccgtggtgttct

SEQ ID NO: 21 - DNA, 20 bp, synthetic construct
  misc_feature 1..20: note = OsUbi1-F-q
  source 1..20: mol_type = other DNA
atggagctgctgctgttcta

SEQ ID NO: 22 - DNA, 20 bp, synthetic construct
  misc_feature 1..20: note = OsUbi1-F-q
  source 1..20: mol_type = other DNA
ttcttccatgctgctctacc

SEQ ID NO: 23 - RNA, 43 nt, synthetic construct
  misc_feature 1..43: note = 5' adapter
  source 1..43: mol_type = other RNA
cgactggagcacaggacactgacatggactgaaggagtagaaa

SEQ ID NO: 24 - DNA, 27 bp, synthetic construct
  misc_feature 1..27: note = Oligo(dT) adapter
  source 1..27: mol_type = other DNA
attctagaggccgaggcggccgacatg

SEQ ID NO: 25 - DNA, 28 bp, synthetic construct
  misc_feature 1..28: note = GeneRacer 5'
  source 1..28: mol_type = other DNA
aggacactgacatggactgaaggagtag

SEQ ID NO: 26 - DNA, 27 bp, synthetic construct
  misc_feature 1..27: note = GeneRacer 3'
  source 1..27: mol_type = other DNA
attctagaggccgaggcggccgacatg

SEQ ID NO: 27 - DNA, 23 bp, synthetic construct
  misc_feature 1..23: note = GSP F
  source 1..23: mol_type = other DNA
gcgtcgacctcctcaaggtgtga

SEQ ID NO: 28 - DNA, 29 bp, synthetic construct
  misc_feature 1..29: note = GSP R
  source 1..29: mol_type = other DNA
gcaactgagaagcagtttgcatcagttgc

SEQ ID NO: 29 - DNA, 30 bp, synthetic construct
  misc_feature 1..30: note = OsU3pro_Hind3_F
  source 1..30: mol_type = other DNA
cccaagcttaaggaatctttaaacatacga

SEQ ID NO: 30 - DNA, 35 bp, synthetic construct
  misc_feature 1..35: note = gRNA:Ter (Xba1)_R
  source 1..35: mol_type = other DNA
tgctctagaaaaacaaaaaagcaccgactcggtgc

SEQ ID NO: 31 - DNA, 40 bp, synthetic construct
  misc_feature 1..40: note = OsU3_NF-NA5_3'UTR_sgRNA_F
  source 1..40: mol_type = other DNA
cgccggtggcaattcatcctgttttagagctagaaatagc

SEQ ID NO: 32 - DNA, 37 bp, synthetic construct
  misc_feature 1..37: note = OsU3P_NF-YA5_3'UTR _sgRNA_R
  source 1..37: mol_type = other DNA
aggatgaattgccaccggcggccacggatcatctgca

SEQ ID NO: 33 - DNA, 40 bp, synthetic construct
  misc_feature 1..40: note = OsU3_miR169a_sgRNA_F
  source 1..40: mol_type = other DNA
ccctgctcctcatgtaaggcgttttagagctagaaatagc

SEQ ID NO: 34 - DNA, 37 bp, synthetic construct
  misc_feature 1..37: note = OsU3P_miR169a_R
  source 1..37: mol_type = other DNA
gccttacatgaggagcaggggccacggatcatctgca

SEQ ID NO: 35 - DNA, 25 bp, synthetic construct
  misc_feature 1..25: note = pENTR_miR169a
  source 1..25: mol_type = other DNA
caccctgaactcatcattcttctcc

SEQ ID NO: 36 - DNA, 21 bp, synthetic construct
  misc_feature 1..21: note = pENTR_miR169a
  source 1..21: mol_type = other DNA
atagaggaggtagtacttatc

SEQ ID NO: 37 - DNA, 21 bp, Oryza sativa
  source 1..21: mol_type = genomic DNA
cagccaaggatgacttgccga

SEQ ID NO: 38 - DNA, 215 bp, Oryza sativa
  source 1..215: mol_type = genomic DNA
aaattaagcagctagccgggaatcgccggcggcctgacattgggatcggaggccatggtgcagccaaggatgacttgccgatcgatcgatctatctatgaagctaagctagctggccatggatccatccatcaattggcaagttgttcttggctacatcttggcccctgctcctcatgtaaggccggcctgtggcgacgacgatcgaaggcgagc

SEQ ID NO: 39 - DNA, 23 bp, synthetic construct
  misc_feature 1..23: note = gRNA for Osa-miR169a
  source 1..23: mol_type = other DNA
ccctgctcctcatgtaaggccgg

SEQ ID NO: 40 - DNA, 1587 bp, Oryza sativa
  source 1..1587: mol_type = genomic DNA
agatagattgagctaagcacacacacacactgcatcagtaccttggtttggttgatttctctgagtttgtgttcttggcatcatcatcattattagccatgagaagtgtaggtggcttggtgttctgaattagtttcagtgttctctttgcacttccatgtgttgcacttgcatcagctatagttccaagaaattgtgtttttgatgatataggcttctctgtctccatttctgcatatgtagtttgctttcttgggctgtggtttcaaacttccaagcttttcttccctctcatggaagaattttccgttcttgcaggcagctctgagcttccaagatcacggccatgggcttcaggcgatcaccggccgtggcggcaatggcggcgccgccgctcacgcattgccatggtgggctggagccggagccggagctggatcccaaacactgcttggaacaggaggaggagaagaatcattctgccaattaagcaacacaatcatggaggacacccgaatcctgcaaaaccaccaccaccagatcctcgccgccggccggcagctgcagcagcgccaccacttcccggcaatgccgccggagcgacaccaccaccctcctcctccagctcctggaagccctgccatgaagttcccaatcatctcaggtgactctgatcttggcaaagatctgaagttccatgagtcctccgcgccgaccatcgccgcgtactcgccattgcaggagtaccaaggacactttgagctagcccttggccactccatggtttgcaccaacttctgcaactctgaacaaagctatggtgtttactccccctatggagctcaaaccatggctgggaggatgctgctgccgccggcgatcgccaccgacgtgggtccgatctacgtcaacgcgaagcagttcaacggcatcatccggcggcggctggcgcgcgccaaggcggagcgggagcaccgggtttcccggagccggaagccgtacctccacgagtcgcgccaccgccacgccatgcgccgcgcgcggggcagcggcggccgcttcctcaacaccaagaacgcctcctccgccgccgccgcggccgccgacgcggcgccggtgagctccggtggcggcgaccacggggcgagcaacaagagctcgtcggcgtcggaggcgacgcgcgtgtacgacgacgacgacgacatgggcgcgggcggcggcggcgacggcggcgacttccaccacgcgatgggtcacctccgctcgccggcgttcttcccgtcgctggccgcgatgatggacggcggcggcggcggcggcgaggggaagtgggcgaccgcgacgcctcaccatggctgccgcgtcgacctcctcaaggtgtgaccgccgccgctggccgtcgccggcgaacctcgccggcgccggtggcaattcatccttggctgtagtagtagtagtagtagtagactagtagtgtatgcgcaatggaaatcaatttcatgcaactgatgcaaactgcttctcagttgctcatgtaaaaaggactagaatttgattttattttgatgcagatttgtttaaatctag

SEQ ID NO: 41 - RNA, 20 nt, synthetic construct
  misc_feature 1..20: note = gRNA for NF-YA5
  source 1..20: mol_type = other RNA
cgccggtggcaattcatcct

SEQ ID NO: 42 - RNA, 84 nt, Oryza sativa
  source 1..84: mol_type = genomic RNA
aaggtgtgaccgccgccgctggccgtcgccggcgaacctcgccggcgccggtggcaattcatccttggctgtagtagtagtagt

SEQ ID NO: 43 - RNA, 21 nt, Oryza sativa
  source 1..21: mol_type = genomic RNA
agccgttcagtaggaaccgac

SEQ ID NO: 44 - DNA, 41 bp, synthetic construct
  misc_feature 1..41: note = OsNF-YA5-3'UTR
  source 1..41: mol_type = other DNA
aacctcgccggcgccggtggcaattcatccttggctgtagt

SEQ ID NO: 45 - DNA, 39 bp, synthetic construct
  misc_feature 1..39: note = OsNF-YA5-3'UTR
  source 1..39: mol_type = other DNA
aacctcgccggcgccggtggcaattcatttggctgtagt

SEQ ID NO: 46 - DNA, 33 bp, synthetic construct
  misc_feature 1..33: note = OsNF-YA5-3'UTR
  source 1..33: mol_type = other DNA
aacctcgccggcgccggtggccttggctgtagt

SEQ ID NO: 47 - DNA, 19 bp, Oryza sativa
  source 1..19: mol_type = genomic DNA
ccctgctcctcatggccgg

SEQ ID NO: 48 - DNA, 14 bp, Oryza sativa
  source 1..14: mol_type = genomic DNA
ccctgctccgccgg

SEQ ID NO: 49 - DNA, 41 bp, Oryza sativa
  source 1..41: mol_type = genomic DNA
aacctcgccggcgccggtggcaatgcaatgcaatttctagc

SEQ ID NO: 50 - DNA, 25 bp, Oryza sativa
  source 1..25: mol_type = genomic DNA
cccctgctcctcacggcctgcctgg